(19) United States
(12) Patent Application Publication
(10) Pub. No.: US 2005/0147575 A1
(43) Pub. Date: Jul. 7, 2005

(54) Microbicidal agent and composition for cosmetic treatment containing it

(75) Inventor: Rainer Muller, Leopoldshafen (DE)

Correspondence Address: NIXON & VANDERHYE, PC, 1100 N GLEBE ROAD, 8TH FLOOR, ARLINGTON, VA, 22201-4714, US

(73) Assignee: L'OREAL

(21) Appl. No.: 10/348,321

(22) Filed: Jan. 22, 2003

Related U.S. Application Data
(60) Provisional application No. 10/360,763, filed on Jan. 22, 2002.

(30) Foreign Application Priority Data
Jan. 7, 2002 (FR) 02 00125

(51) Int. Cl. A61K 7/06; A61K 7/11

(52) U.S. Cl. 424/70.17

(57) ABSTRACT

The present invention relates to a microbicidal agent comprising anisic acid, one of its salts or one of its C₁₋₄ alkyl esters, at least one polyol and at least one cationic surfactant. This microbicidal agent makes it possible in particular to prevent the proliferation of bacteria and fungi such as Aspergillus niger, and may be used in cosmetics. The invention also relates to a composition for the cosmetic treatment of keratinous materials, such as a shampoo, containing it.

The present invention relates to a microbicidal agent and to a composition for treating keratinous materials containing it.

Contaminants such as bacteria and fungi develop over time in cosmetic compositions. These contaminants may be the source of allergies.

To avoid this type of problem linked to these contaminants, it is well known to use preservatives such as propylparaben, benzoic acid, cresols or formaldehyde, but they themselves can cause allergies.

A need therefore exists for cosmetic products, such as shampoos, which do not contain preservatives or contain little preservative so as to substantially limit the risks of allergies.

In its research relating to a novel microbicidal agent, the Applicant discovered, surprisingly, that substantial microbiological contamination was avoided by combining anisic acid with a polyol and a cationic surfactant.

The subject of the present invention is therefore a microbicidal agent comprising anisic acid, one of its salts or one of its C₁-C₄ alkyl esters, at least one polyol and at least one cationic surfactant.

The subject of the invention is also a composition for cosmetic treatment containing it.

Other objects, characteristics, aspects and advantages of the invention will appear more clearly on reading the description and the various examples which follow.

According to the invention, the microbicidal agent comprises anisic acid, one of its salts or one of its C₁-C₄ alkyl esters, at least one polyol and at least one cationic surfactant.

The anisic acid used in the present invention may be used as it is or may be present in the form of an alkali or alkaline-earth metal salt, an ammonium salt or a salt with an organic amine, or in the form of a C₁₋₄ alkyl ester. The alkyl group of the ester may be linear or branched and there may be mentioned, by way of examples, methyl, ethyl, n-propyl, isopropyl, n-butyl and tert-butyl groups.

By way of examples of anisic acid salt or ester, there may be mentioned in particular sodium anisate, potassium anisate, methyl anisate, ethyl anisate, propyl anisate or butyl anisate.

Preferably, the microbicidal agent contains anisic acid.

Anisic acid, the salt or the ester of this acid is in particular contained in a quantity from 1 to 30% by weight, preferably from 4 to 20% by weight relative to the total weight of the microbicidal agent, or in a quantity ranging up to 1% by weight, preferably in a quantity from 0.1 to 0.4% by weight relative to the total weight of a composition containing the microbicidal agent.

The expression polyol is understood to mean an alcohol which has a hydrocarbon chain comprising at least 4 carbon atoms, preferably from 4 to 50 carbon atoms, and carrying at least two hydroxyl groups, preferably from 2 to 10 hydroxyl groups. The polyols generally used in the present invention have a weight-average molecular mass of less than or equal to 1 000, preferably of between 90 and 500.

As polyol which can be used according to the present invention, there may be mentioned in particular a polyalkylene glycol, such as polyethylene glycols, in particular PEG-4, PEG-6 and PEG-8, pentanediols and in particular 1,2-pentanediol, sorbitol or mixtures thereof; 1,2-pentanediol, alone or in the form of a mixture with other polyols, being particularly preferred.

The microbicidal agent according to the invention preferably comprises up to 50% by weight, better still from 10 to 30% by weight of at least one polyol as mentioned above, relative to the total weight of microbicidal agent. The polyol(s) represent(s) up to 10% by weight, better still 5 to 10% by weight of the total weight of a composition containing the microbicidal agent.

The microbicidal agent according to the invention also comprises one or more cationic surfactants well known per se, such as the salts of optionally polyoxyalkylenated primary, secondary or tertiary fatty amines, quaternary ammonium salts, or mixtures thereof.

The fatty amines are in particular stearamidopropyldimethylamine or behenylamidopropyldimethylamine.

By way of quaternary ammonium salts, there may be mentioned, for example, in particular:
those which have the following general formula (I): 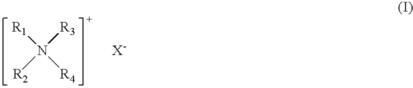
   in which the symbols R₁ to R₄, which may be identical or different, represent a linear or branched aliphatic group comprising from 1 to 30 carbon atoms, or an aromatic group such as aryl or alkylaryl. The aliphatic groups may comprise heteroatoms such as in particular oxygen, nitrogen, sulphur, phosphorus and halogen atoms. The aliphatic groups are, for example, chosen from alkyl, alkoxy, (C₂-C₆)polyoxyalkylene, alkylamide, (C₁₂-C₂₂)alkylamido(C₂-C₆)alkyl, (C₁₂-C₂₂)alkyl acetate and hydroxyalkyl groups comprising from about 1 to 30 carbon atoms; X⁻ is an anion chosen from the set consisting of halides, phosphates, acetates, lactates, (C₂-C₆)alkyl sulphates, alkyl or alkylaryl sulphonates;
the quaternary ammonium salts of imidazoline such as, for exmaple, those of the following formula (II): 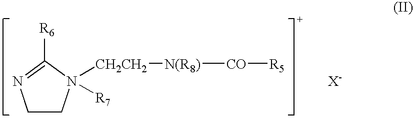
   in which R₅ represents an alkenyl or alkyl group comprising from 8 to 30 carbon atoms, for example derived from tallow fatty acids, R₆ represents a hydrogen atom, a C₁-C₄ alkyl group or an alkenyl or alkyl group comprising from 8 to 30 carbon atoms, R₇ represents a C₁-C₄ alkyl group, R₈ represents a hydrogen atom, a C₁-C₄ alkyl group, X⁻ is an anion chosen from the set consisting of halides, phosphates, acetates, lactates, alkyl sulphates, alkyl or alkylaryl sulphonates. Preferably, R₅ and R₆ denote a mixture of alkenyl or alkyl groups comprising from 12 to 21 carbon atoms, for example derived from tallow fatty acids, R₇ denotes a methyl group, R₈ denotes a hydrogen atom. Such a product is, for example, that known under the INCI name Quarternium-27 (CTFA dictionary, 8th edition, 2000, volume 2) and marketed under the name REWOQUAT® W 75 by the company REWO.
the quaternary diammonium salts of formula (III): 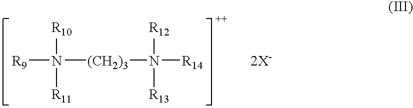
   in which R₉ denotes an aliphatic group comprising about from 16 to 30 carbon atoms, R₁₀, R₁₁, R₁₂, R₁₃ and R₁₄, which are identical or different, each represent a hydrogen atom or, an alkyl group comprising from 1 to 4 carbon atoms, and X⁻ is an anion chosen from the set consisting of halides, acetates, phosphates, nitrates and methyl sulphates. Such quaternary diammonium salts comprise in particular propanetallowdiammonium dichloride.
the quaternary ammonium salts containing at least one ester functional group, such as those of the following formula (IV): 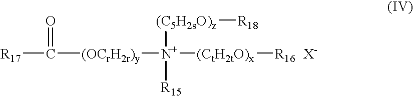
in which:
R₁₅ is chosen from C₁-C₆ alkyl groups and C₁-C₆ hydroxyalkyl or dihydroxyalkyl groups;
R₁₆ is chosen from:
   the radical 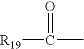
   the saturated or unsaturated, linear or branched C₁-C₂₂ hydrocarbon groups R₂₀,
   a hydrogen atom,
R₁₇ is chosen from:
   the radical 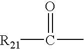
   the saturated or unsaturated, linear or branched C₁-C₆ hydrocarbon groups R₂₂,
   a hydrogen atom,
R₁₇, R₁₉ and R₂₁, which are identical or different, are chosen from saturated or unsaturated, linear or branched C₇-C₂₁ hydrocarbon groups;
r, s and t, which are identical or different, are integers ranging from 2 to 6;
y is an integer ranging from 1 to 10;
x and z, which are identical or different, are integers ranging from 0 to 10;
X⁻ is a simple or complex, organic or inorganic anion;
provided that the sum x+y+z is equal to 1 to 15, that when x is equal to 0, then R₁₆ denotes R₂₀ and that when z is equal to 0, then R₁₈ denotes R₂₂.

The alkyl groups R₁₅ may be linear or branched, and more particularly linear.

Preferably, R₁₅ denotes a methyl, ethyl, hydroxyethyl or dihydroxypropyl group, and more particularly a methyl or ethyl group.

Advantageously, the sum x+y+z is equal to 1 to 10.

When R₁₆ is a hydrocarbon group R₂₀, it may be long and may have from 12 to 22 carbon atoms, or may be short and may have from 1 to 3 carbon atoms.

When R₁₈ is a hydrocarbon group R₂₂, it preferably has 1 to 3 carbon atoms.

Advantageously, R₁₇, R₁₉ and R₂₁, which are identical or different, are chosen from saturated or unsaturated, linear or branched C₁₁-C₂₁ hydrocarbon groups, and more particularly from saturated or unsaturated, linear or branched C₁₁-C₂, alkyl and alkenyl groups.

Preferably, x and z, which are identical or different, are equal to 0 or 1.

Advantageously, y is equal to 1.

Preferably, r, s and t, which are identical or different, are equal to 2 or 3, and more particularly still are equal to 2.

The anion is preferably a halide (chloride, bromide or iodide) or an alkyl sulphate, more particularly methyl sulphate. Methane sulphonate, phosphate, nitrate, tosylate, an anion derived from an organic acid such as acetate or lactate, or any other anion compatible with ammonium containing an ester functional group may however be used.

The anion X⁻ is more particularly still chloride or methyl sulphate.

In the composition according to the invention there are more particularly used the ammonium salts of formula (IV) in which:
R₁₅ denotes a methyl or ethyl group,
x and y are equal to 1;
z is equal to 0 or 1;
r, s and t are equal to 2;
R₁₆ is chosen from:
   the radical 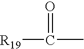
   the methyl, ethyl or C₁₄-C₂₂ hydrocarbon groups;
   a hydrogen atom;
R₁₈ is chosen from:
   the radical 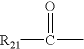
   a hydrogen atom;
R₁₇, R₁₉ and R₂₁, which may be identical or different, are chosen from saturated or unsaturated, linear or branched C₁₃-C₁₇ hydrocarbon groups and preferably from saturated or unsaturated, linear or branched C₁₃-C₁₇ alkyl and alkenyl groups.

Advantageously, the hydrocarbon groups are linear.

There may be mentioned, for example, the compounds of formula (IV) such as the salts (chloride or methyl sulphate in particular) of diacyloxyethyldimethylammonium, diacyloxyethylhydroxyethylmethylammonium, monoacyloxyethyldihydroxyethylmethylammonium, triacyloxyethylmethylammonium, monoacyloxyethylhydroxyethyldimethylammonium and mixtures thereof. The acyl groups preferably have 14 to 18 carbon atoms and are obtained more particularly from a vegetable oil such as palm or sunflower oil. When the compound contains several acyl groups, the latter may be identical or different.

These products are obtained, for example, by direct esterification of optionally oxyalkylenated triethanolamine, triisopropanolamine, alkyldiethanolamine or alkyldiisopropanolamine with fatty acids or with mixtures of fatty acids of plant or animal origin or by transesterification of their methyl esters. This esterification is followed by quaternization using an alkylating agent such as an alkyl (preferably methyl or ethyl) halide, a dialkyl (preferably methyl or ethyl) sulphate, methyl methanesulphonate, methyl para-toluenesulphonate, glycol chlorohydrin or glycerol chlorohydrin.

Such compounds are, for example, marketed under the names DEHYQUART® by the company COGNIS, STEPANQUAT® by the company STEPAN, NOXAMIUM® by the company CECA, REWOQUAT® WE 18 by the company REWO-WITCO.

The composition according to the invention may preferably contain a mixture of quaternary ammonium mono-, di- and triester salts with a majority, by weight, of diester salts.

As the mixture of ammonium salts, there may be used, for example, the mixture containing 15 to 30% by weight of acyloxyethyldihydroxyethylmethylammonium methyl sulphate, 45 to 60% of diacyloxyethylhydroxyethylmethylammonium methyl sulphate and 15 to 30% of triacyloxyethylmethylammonium methyl sulphate, the acyl groups having from 14 to 18 carbon atoms and being obtained from optionally partially hydrogenated palm oil.

The ammonium salts containing at least one ester functional group which are disclosed in U.S. Pat. No. 4,874,554 and U.S. Pat. No. 4,137,180 may also be used.

Among the quaternary ammonium salts mentioned above, the use of those corresponding to the formula (I) is preferred. There may be mentioned in particular tetraalkylammonium chlorides such as, for example, dialkyldimethylammonium or alkyltrimethylammonium chlorides in which the alkyl group comprises from about 12 to 22 carbon atoms, in particular behenyltrimethylammonium, distearyldimethylammonium, cetyltrimethylammonium or benzyldimethylstearylammonium chlorides; palmitylamidopropyltrimethylammonium chloride or stearamidopropyldimethyl(myristyl acetate)ammonium chloride marketed under the name CERAPHYL® 70 by the company VAN DYK, or hydroxyethylhydroxycetyldimonium chloride (INCI name) marketed under the name DEHYQUART® E-CA by the company COGNIS, dihydroxypropyl-PEG-5-linoleammonium chloride (INCI name) marketed under the name MONAQUAT® SL-5 by the company UNIQEMA and linoleamidopropyl-PG-dimmonium chloride-phosphate (INCI name) marketed under the name PHOSPHOLIPID® EFA by the company UNIQEMA.

The cationic surfactant particularly used in the microbicidal agent of the invention is preferably a quaternary ammonium salt of imidazoline corresponding to the formula (II) such as quaternium-27.

The microbicidal agent according to the invention preferably contains at least one cationic surfactant in a quantity from 20 to 80% by weight, preferably from 30 to 60% by weight relative to the total weight of the microbicidal agent, or the surfactants are contained in a quantity from 0.05 to 10% by weight, preferably from 0.1 to 5% by weight relative to the total weight of a composition containing the microbicidal agent.

A particularly preferred microbicidal agent of the present invention comprises anisic acid, a pentanediol and a quaternium-27 in the proportions described above.

The microbicidal agent according to the invention has in particular a specific action against bacteria and fungi such as Aspergillus niger and thus prevents their proliferation.

Another subject of the invention consists in the use of the microbicidal agent as described above in cosmetics, in particular for decontaminating a cosmetic composition such as a shampoo.

This microbicidal agent may then be used in a quantity of between 0.1 and 20% by weight, preferably between 1 and 10% by weight relative to the total weight of the cosmetic composition.

The composition for the cosmetic treatment of keratinous materials according to the invention comprises, in a cosmetically acceptable medium, at least one microbicidal agent as described above, in a quantity of between 0.1 and 20% by weight, preferably between 1 and 10% by weight relative to the total weight of the composition for cosmetic treatment.

The expression cosmetically acceptable medium is understood to mean a medium compatible with all keratinous materials such as the skin, the nails, the hair, the eyelashes, the eyebrows, the lips and any other cutaneous region of the body and of the face.

The cosmetically acceptable aqueous medium may consist of water or of a mixture of water and of one or more cosmetically acceptable solvents such as C₁-C₄ lower alcohols, for example ethanol, isopropanol, tert-butanol, n-butanol; alkylene glycols such as propylene glycol; polyol ethers; C₅-C₁₀ alkanes; acetone, methyl ethyl ketone; C₁-C₄ alkyl acetates such as methyl acetate, ethyl acetate, butyl acetate; dimethoxyethane, diethoxyethane; or one of the mixtures thereof.

The composition may also contain one or more anionic, nonionic or amphoteric surfactants well known in the art.

As anionic surfactants which can be used in the present invention, there may be mentioned in particular the salts, in particular alkali metal, salts such as sodium salts, ammonium salts, amine salts, amino alcohol salts or alkaline-earth metal salts, for example, magnesium salts of the following types: alkyl sulphates, alkyl ether sulphates, alkylamido ether sulphates, alkylaryl polyether sulphates, monoglyceride sulphates, alkyl sulphonates, alkylamidesulphonates, alkylaryl sulphonates, α-olefin sulphonates, paraffin sulphonates, alkyl sulphosuccinates, alkyl ether sulphosuccinates, alkylamidesulphosuccinates, alkyl sulphoacetates, acyl sarcosinates, and N-acylglutamates, the alkyl or acyl groups of all these different compounds containing from 6 to 24 carbon atoms, and the aryl group preferably denoting a phenyl or benzyl group. It is also posisble to use the esters of C₆-C₂₄ alkyl and of polyglycosidecarboxylic acids such as alkyl glucoside citrates, alkyl polyglycoside tartrates, and alkyl polyglycoside sulphosuccinates; alkyl sulphosuccinamates, acyl isethionates and N-acyl taurates, the alkyl or acyl groups of all these compounds comprising from 12 to 20 carbon atoms. Among the anionic surfactants which can still be used, there may also be mentioned acyl lactylates in which the acyl group comprises from 8 to 20 carbon atoms.

In addition there may also be mentioned alkyl-D-galactosideuronic acids and salts thereof, as well as the polyoxyalkylenated carboxylic (C₆-C₂₄)alkyl ether acids, the polyoxyalkylenated carboxylic (C₆-C₂₄)alkyl(C₆-C₂₄)aryl ether acids, the polyoxyalkylenated carboxylic (C₆-C₂₄)alkyl amidoether acids and salts thereof, in particular those containing from 2 to 50 ethylene oxide groups and mixtures thereof.

The nonionic surfactants that are suitable in the invention are compounds which are well known per se (in this respect see especially the “Handbook of Surfactants” by M. R. PORTER, published by Blackie & Son (Glasgow and London), 1991, pp. 116-178). They can thus be chosen especially from alcohols, alpha-diols, (C₁-C₂₀) alkylphenols or polyethoxylated, polypropoxylated or polyglycerolated fatty acids which have a fatty chain containing, for example, 8 to 18 carbon atoms, it being possible for the number of ethylene oxide or propylene oxide groups to range especially from 2 to 50 and it being possible for the number of glycerol groups to range especially from 2 to 30. The copolymers of ethylene oxide and propylene oxide and the condensates of ethylene oxide and propylene oxide with fatty alcohols may also be mentioned; the polyethoxylated fatty amides preferably containing from 2 to 30 mol of ethylene oxide, the polyglycerolated fatty amides on average containing 1 to 5 glycerol groups and in particular 1.5 to 4; the polyethoxylated fatty amines preferably containing 2 to 30 mol of ethylene oxide; the ethoxylated fatty acid esters of sorbitan containing from 2 to 30 mol of ethylene oxide; the fatty acid esters of sucrose, the fatty acid esters of polyethylene glycol, (C₆-C₂₄)alkylpolyglycosides, the N-(C₆-C₂₄)alkylglucamine derivatives, amine oxides such as the oxides of (C₁₀-C₁₄)alkylamines or the N-(C₁₀-C₁₄)acylaminopropylmorpholine oxides; and mixtures thereof.

The amphoteric surfactants that are suitable in the present invention may be especially (nonlimiting list) derivatives of aliphatic secondary or tertiary amines in which the aliphatic group is a linear or branched chain containing 8 to 22 carbon atoms and containing at least one water-solubilizing anionic group such as, for example, a carboxylate, sulphonate, sulphate, phosphate or phosphonate group; (C₈-C₂₀)alkylbetaines, sulphobetaines, (C₈-C₂₀)alkylamido(C₆-C₈)alkyl betaines or (C₈-C₂₀)alkylamido(C₆-C₈)alkylsulphobetaines; and mixtures thereof may further be mentioned.

Among the amine derivatives there may be mentioned the products sold under the name MIRANOL®, as described in U.S. Pat. No. 2,528,378 and U.S. Pat. No. 2,781,354 and classified in the CTFA dictionary, 3rd edition, 1982, under the names Amphocarboxyglycinate and Amphocarboxypropionate of the respective structures (1) and (2):
Rₐ—CONHCH₂CH₂—N(R_{b})(R_{C})(CH₂COO⁻) (1)
in which:
Rₐ represents an alkyl group derived from an acid Rₐ—COOH present in hydrolysed copra oil, a heptyl, nonyl or undecyl group,
R_{b} represents a beta-hydroxyethyl group and
R_{c} represents a carboxymethyl group;
   and
   R_{a′}—CONHCH₂CH₂—N(B)(C) (2)
   in which:
B represents —CH₂CH₂OX′,
C represents —(CH₂)_{z}—Y′, with z=1 or 2,
X′ represents the —CH₂CH₂—COOH group or a hydrogen atom,
Y′ represents —COOH or the group —CH₂—CHOH—SO₃H,
R_{a′} represents an alkyl group of an acid Ra′-COOH present in copra oil or in hydrolysed linseed oil, an alkyl group, especially C₁₇, and its iso form or an unsaturated group C₁₇.

These compounds are classified in the CTFA dictionary, 5th edition, 1993, under the names disodium cocoamphodiacetate, disodium lauroamphodiacetate, disodium caprylamphodiacetate, disodium capryloamphodiacetate, disodium cocoamphodipropionate, disodium lauroamphodipropionate, disodium caprylamphodipropionate, disodium capryloamphodipropionate, lauroamphodipropionic acid, cocoamphodipropionic acid.

By way of example, there may be mentioned the cocoamphodiacetate marketed under the trade name MIRANOL® C2M concentrate by the company RHODIA.

The compositions according to the invention may also contain one or more conventional additives well known in the art, such as cationic, anionic, nonionic or amphoteric polymers, silicones, paraffins, esters, polymeric stabilizers, natural or synthetic polymeric thickeners, which are anionic, amphoteric, zwitterionic, nonionic or cationic, associative or otherwise, nonpolymeric thickeners such as acids or electrolytes, opacifiers, perfumes, vegetable, mineral and/or synthetic oils, colorants, organic or inorganic particles, preservatives, pH-stabilizing agents.

Persons skilled in the art will be careful to choose the possible additives and their quantity so that they do not adversely affect the properties of the compositions of the present invention.

These additives are present in the composition according to the invention in a quantity ranging from 0 to 20% by weight relative to the total weight of the composition.

The cosmetic compositions according to the invention may be provided in the form of fluid or thickened liquids, or gels, creams, foams, simple emulsions or multiple emulsions.

They may be used, for example, as shampoos, rinse-off treatments, deep treatment masks, lotions or creams for treating the scalp.

According to a preferred embodiment of the invention, the composition may be used as a shampoo.

The present invention also relates to a method for the cosmetic treatment of keratinous materials, in particular the hair, which consists in applying to the hair an effective quantity of a composition for cosmetic treatment as described above, and in optionally rinsing off after a possible exposure time.

The following examples illustrate the present invention and should not be considered in any way as limiting the invention.

### EXAMPLES

### Example 1

Shampoo formulations were prepared from the ingredients indicated in Table 1 below:

TABLE 1

| Composition | 1 | A | B | C |
|---|---|---|---|---|
| Glycerin | 7.00 | 7.00 | 7.00 | 7.00 |
| 1,2-Pentanediol | 0.10 | 0.10 | 0.10 | — |
| Anisic acid | 0.20 | 0.20 | — | 0.20 |
| Quaternium-27 (75% A.S.)⁽¹⁾ | 0.60 | — | 0.60 | — |
| Guar-hydroxypropyltrimonium | — | 0.05 | — | — |
| chloride | | | | |
| PEG-150 distearate | 4.20 | 3.50 | 5.00 | 3.75 |
| Decyl glucoside (50% A.S.) | 30.00 | 30.00 | 30.00 | 30.00 |
| Sodium chloride | 3.00 | 3.00 | 3.00 | 3.00 |
| Citric acid | 2.40 | 2.40 | 2.40 | 2.40 |
| Sodium hydroxide | 0.92 | 0.94 | 0.95 | 1.05 |
| Water qs | 100 | 100 | 100 | 100 |
| pH | 5.2 | 5.2 | 5.2 | 5.2 |

⁽¹⁾Marketed by REWO under the name REWOQUAT ® W75

Composition 1 is a composition according to the invention. Compositions A, B and C contain only certain constituents of the microbicidal agent.

To determine the bactericidal efficacy of the microbicidal agent according to the present invention in a shampoo, the test as described in European Pharmacopoeia—supplement 2001, pages 293-294, was used. This test made it possible to observe the variation in the number of microorganisms, here the fungi Aspergillus niger over time in the four compositions.

Each of the compositions 1, A, B and C were contaminated with a quantity of Aspergillus niger fungi in suspension in a solution containing 9 g/l of sodium chloride and 0.5 g/l of polysorbate 80, the volume of the suspension not exceeding 1% of the volume of each composition.

After having vigorously mixed the inoculated compositions in order to obtain a uniform distribution of the microorganisms therein, 1 ml of each inoculated composition was removed and the number of microorganisms was determined, this first measurement corresponding to t=0 day, and then they were stored protected from light at a temperature of 20 to 25° C.

The number of microorganisms was then determined after 7 days and 14 days. The results are indicated in Table 2 below.

TABLE 2
Aspergillus niger (number of microorganisms/ml of composition)

| 0 day | 5.1 × 10⁵ | 2.8 × 10⁵ | 5.1 × 10⁵ | 1.4 × 10⁵ |
|---|---|---|---|---|
| 7 days | 3.0 × 10² | 9.0 × 10⁴ | 4.3 × 10⁵ | 5.9 × 10⁴ |
| 14 days | <100 | 1.2 × 10⁴ | 2.9 × 10⁵ | 6.5 × 10³ |

A very significant reduction in the number of Aspergillus niger fungi was observed in the composition according to the invention, compared with compositions A, B and C.

### Examples 2 à 4

Other compositions according to the invention have been prepared from ingredients presented in the below table 2, proportions being indicated in weight %.

TABLE 3

| Examples | 2 | 3 | 4 |
|---|---|---|---|
| Anisic acid | 0.2 | 0.2 | 0.2 |
| 1,2-pentanediol | 0.1 | 0.1 | 0.1 |
| Quaternium-27 (75% M.A.)⁽¹⁾ | 0.45 | 0.45 | 0.45 |
| | (M.A.) | (M.A.) | (M.A.) |
| PEG-150 distearate | 4.4 | 4 | 3.5 |
| PEG-4 | 7 | | |
| PEG-8 | | 7 | |
| Sorbitol (70% M.A.) | | | 7 |
| | | | (M.A.) |
| Decylglucoside (50% M.A.) | 15 | 15 | 15 |
| | (M.A.) | (M.A.) | (M.A.) |
| Sodium chloride | 3 | 3 | 3 |
| Citric acid | 2.4 | 2.4 | 2.4 |
| Water qsp | 100 | 100 | 100 |
| Sodium hydroxyde qs pH | 5.2 | 5.2 | 5.2 |

⁽¹⁾ Marketed by REWO under the name REWOQUAT ® W75

1. Microbicidal agent comprising anisic acid, one of its salts or one of its C₁₋₄ alkyl esters, at least one polyol having a hydrocarbon chain with at least 4 carbon atoms and at least two hydroxy groups, and at least one cationic surfactant:

2. Microbicidal agent according to claim 1, characterized in that the anisic acid salt is chosen from an alkali or alkaline-earth metal salt, an ammonium salt or a salt with an organic amine.

3. Microbicidal agent according to claim 1, characterized in that the polyol has a hydrocarbon chain comprising from 4 to 50 carbon atoms and carrying 2 to 10 hydroxyl groups, and has a weight-average molecular mass of less than or equal to 1 000.

4. Microbicidal agent according to claim 3, characterized in that the polyol is chosen from polyalkylene glycols, pentanediols, sorbitol or mixtures thereof.

5. Microbicidal agent according to claim 1, characterized in that the cationic surfactant is chosen from:
those which have the following general formula (I): 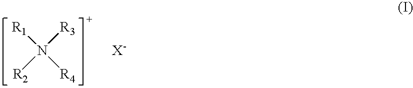
in which the symbols R₁ to R₄, which may be identical or different, represent a linear or branched aliphatic group comprising from 1 to 30 carbon atoms, or an aromatic group such as aryl or alkylaryl; X⁻ is an anion chosen from the set consisting of halides, phosphates, acetates, lactates, (C₂-C₆)alkyl sulphates, alkyl or alkylaryl sulphonates;
the quaternary ammonium salts of imidazoline;
the quaternary diammonium salts of formula (III): 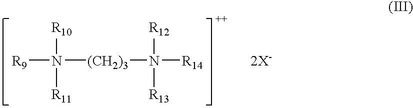
in which R₉ denotes an aliphatic group comprising about from 16 to 30 carbon atoms, R₁₀, R₁₁, R₁₂, R₁₃ and R₁₄, which are identical or different, each represent a hydrogen atom or an alkyl group comprising from 1 to 4 carbon atoms, and X⁻ is an anion chosen from the set consisting of halides, acetates, phosphates, nitrates and methyl sulphates; and
the quaternary ammonium salts containing at least one ester functional group.

6. Microbicidal agent according to claim 5, characterized in that the cationic surfactant is quaternium-27.

7. Microbicidal agent according to claim 1, characterized in that the anisic acid, one of its salts or one of its esters is contained in a quantity from 1 to 30% by weight, preferably from 4 to 20% by weight relative to the total weight of the microbicidal agent.

8. Microbicidal agent according to claim 1, characterized in that the anisic acid, one of its salts or one of its esters is contained in a quantity ranging up to 1% by weight, preferably in a quantity from 0.1 to 0.4% by weight relative to the total weight of a composition containing the microbicidal agent.

9. Microbicidal agent according to claim 1, characterized in that the polyol is contained in a quantity ranging up to 50% by weight, preferably in a quantity from 10 to 30% by weight relative to the total weight of the microbicidal agent.

10. Microbicidal agent according to claim 1, characterized in that the polyol is contained in a quantity ranging up to 10% by weight, preferably in a quantity from 5 to 10% by weight relative to the total weight of a composition containing the microbicidal agent.

11. Microbicidal agent according to claim 1, characterized in that the cationic surfactant is contained in a quantity from 20 to 80% by weight, preferably from 30 to 60% by weight relative to the total weight of the microbicidal agent.

12. Microbicidal agent according to claim 1, characterized in that the cationic surfactant is contained in a quantity from 0.05 to 10% by weight, preferably from 0.1 to 5% by weight relative to the total weight of a composition containing the microbicidal agent.

13. Microbicidal agent according to claim 1, characterized in that it comprises anisic acid, a pentanediol and quaternium-27.

14. Microbicidal agent according to claim 1, characterized in that it prevents the proliferation of bacteria and fungi such as Aspergillus niger.

15. Microbicidal agent according to claim 1, characterized in that it is used in cosmetics.

16. Microbicidal agent according to claim 1, characterized in that it is used for decontaminating a cosmetic composition.

17. Composition for the cosmetic treatment of keratinous materials, comprising, in a cosmetically acceptable medium, at least one microbicidal agent according to claim 1.

18. Composition according to claim 17, characterized in that the microbicidal agent is contained in a quantity of between 0.1 and 20% by weight relative to the total weight of the composition.

19. Composition according to claim 17, characterized in that the cosmetically acceptable medium consists of water or of a mixture of water and of one or more cosmetically acceptable solvents.

20. Composition according to claim 19, characterized in that the cosmetically acceptable solvents are chosen from C₁-C₄ lower alcohols, alkylene glycols, polyol ethers, C₅-C₁₀ alkanes, acetone, methyl ethyl ketone, C₁-C₄ alkyl acetates, dimethoxyethane, diethoxyethane, or one of the mixtures thereof.

21. Composition according to claim 17, characterized in that it comprises one or more anionic, nonionic or amphoteric surfactants.

22. Composition according to claim 17, characterized in that it comprises one or more additives such as cationic, anionic, nonionic or amphoteric polymers, silicones, paraffins, esters, polymeric stabilizers, natural or synthetic polymeric thickeners, which are anionic, amphoteric, zwitterionic, nonionic or cationic, associative or otherwise, nonpolymeric thickeners such as acids or electrolytes, opacifiers, perfumes, vegetable, mineral and/or synthetic oils, colorants, organic or inorganic particles, preservatives, pH-stabilizing agents.

23. Composition according to claim 17, characterized in that it is used as a shampoo, rinse-off treatment, deep treatment mask, lotion or cream for treating the scalp.

24. Method of cosmetic treatment, characterized in that it consists in applying to the hair an effective quantity of a composition for cosmetic treatment according to claim 17, and in optionally rinsing after a possible exposure time.
